(19) United States
(12) Patent Application Publication
(10) Pub. No.: US 2025/0235367 A1
(43) Pub. Date: Jul. 24, 2025

(54) URINE COLLECTION SYSTEM INCLUDING A FLOW METER AND RELATED METHODS

(71) Applicant: PUREWICK CORPORATION, Covington, GA (US)

(72) Inventors: Michelle Tourchak, Alpharetta, GA (US); Kristin Watson, Social Circle, GA (US); Jill W. Jones, Atlanta, GA (US); Hollie Trullenque, Atlanta, GA (US); Gina Whitlock, Barrington Hills, IL (US); Mark Weissmann, Grapevine, TX (US); Adam Voigt, Chapin, SC (US); Cecille Canary, Atlanta, GA (US)

(21) Appl. No.: 19/171,983

(22) Filed: Apr. 7, 2025

Related U.S. Application Data
(63) Continuation of application No. 18/584,002, filed on Feb. 22, 2024, now Pat. No. 12,290,485.
Application No. 18/584,002 is a continuation of application No. 17/453,560, filed on Nov. 4, 2021, now Pat. No. 12,070,432.
(60) Provisional application No. 63/112,417, filed on Nov. 11, 2020.

(51) Int. Cl. A61G 9/00; A61M 1/00; G16H 10/60

(52) U.S. Cl. CPC A61G 9/003; A61M 1/64; A61M 1/71; G16H 10/60; A61M 2202/0496; A61M 2205/3331; A61M 2205/3379; A61M 2205/3553; A61M 2205/583

(57) ABSTRACT

Examples relate to devices, systems, and methods for collecting and measuring discharged urine. A urine collection system includes a urine collection device configured to collect urine discharged from a user and a urine collection container in fluid communication with the urine collection device and sized and dimensioned to receive the urine discharged from the user. The urine collection system also includes a flow meter having a communication interface and positioned between the urine collection container and the urine collection device. The flow meter is configured to detect a flow property of the urine at least related to a volume of the urine entering the urine collection container and the communication interface is configured to wirelessly communicate to a computing device at least one of the detected flow property or the volume of the urine including the volume of the urine discharged from the user entering the urine collection container.

### CROSS-REFERENCE TO RELATED APPLICATIONS

This application is a continuation of U.S. application Ser. No. 18/584,002 filed Feb. 22, 2024, which is a continuation of U.S. application Ser. No. 17/453,560 filed Nov. 4, 2021 and issued as U.S. Pat. No. 12,070,432 on Aug. 27, 2024, which claims priority to U.S. Provisional Patent Application No. 63/112,417 filed on Nov. 11, 2020, the disclosure of each of which is incorporated herein, in its entirety, by this reference.

### BACKGROUND

An individual may have limited or impaired mobility such that typical urination processes are challenging or impossible. For example, the individual may have surgery or a disability that impairs mobility. In another example, the individual may have restricted travel conditions such as those experience by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections.

Thus, users and manufacturers of urine collection devices continue to seek new and improved devices, systems, and methods to collect urine.

### SUMMARY

Embodiments disclosed herein are related to urine collection devices and methods of using urine collection devices. In an embodiment, a urine collection system includes a urine collection device configured to collect urine discharged from a user and a urine collection container in fluid communication with the urine collection device and sized and dimensioned to receive the urine discharged from the user. The urine collection system also includes a flow meter having a communication interface and positioned between the urine collection container and the urine collection device. The flow meter is configured to detect a flow property of the urine at least related to a volume of the urine entering the urine collection container and the communication interface is configured to wirelessly communicate to a computing device at least one of the detected flow property or the volume of the urine including the volume of the urine discharged from the user entering the urine collection container.

In an embodiment, a method of determining a volume of urine discharged by a user includes receiving urine discharged from the user in a urine collection device. The method also includes detecting, with a flow meter, a flow property of the urine at least related to a volume of the urine discharged by the user as the urine is passed from the urine collection device to a urine collection container. The method also includes wirelessly transmitting, from a communication interface of the flow meter to a computing device, at least one of the detected flow property or the volume of the urine including the volume of the urine.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a urine collection system including a flow meter, according to an embodiment.

FIG. 1B is an isometric view of the urine collection system of FIG. 1A in use, according to an embodiment.

FIG. 1C is an isometric view of a urine collection container, according to an embodiment.

FIG. 2A is a block diagram of a urine collection system including a flow meter, according to an embodiment.

FIG. 2B is a side view of the urine collection system of FIG. 1B in use, according to an embodiment.

FIG. 2C is a side view of a urine collection system in use, according to an embodiment.

FIG. 3A is a block diagram of a urine collection system including a flow meter, according to an embodiment.

FIG. 3B is an isometric view of the urine collection system of FIG. 3B, according to an embodiment.

FIG. 4 is a flow diagram of a method of determining a volume of liquid discharged by a user.

FIG. 5 is a schematic for a computing device in a flow meter of a urine collection system, according to an embodiment.

### DETAILED DESCRIPTION

Embodiments disclosed herein are related to fluid collection systems including an automated urine output (AUO) measurement, and methods of using the same. The devices and systems disclosed herein are configured to collect fluids from an individual and include a flow meter. The fluids collected by the urine collection devices may include at least one of urine, vaginal discharge, penile discharge, reproductive fluids, blood, sweat, or other bodily fluids. Embodiments of urine collection systems disclosed herein may include a flow meter configured to detect and wirelessly transmit a flow property of the urine to a computing device. The flow property of the urine may be related at least to a volume of the urine exiting the urine collection device and/or entering the urine collection container. In some embodiments, the flow property may include at least one of a volume, a mass, or a velocity of the urine passing through or proximate to the flow meter. For example, the flow property may include a flow rate of the urine over a predetermined period of time. At least one of the flow meter or the computing device may be configured to translate the flow property of the urine detected by the flow meter into one or more volumetric measurement(s) of urine exiting the urine collection device and/or entering the urine collection container. By detecting and wirelessly transmitting the flow property and/or the volume of the urine detected by the flow meter, one or more embodiments of the fluid collection systems described herein may determine, collect, and/or utilize (in real-time) more accurate volumetric flow rates and urine discharge information.

In some examples, the flow property of the urine detected by the flow meter in the urine collection system may allow the fluid collection systems to be used with a variety of patient demographics, including ambulatory patients that need urine flow rates monitored when asleep or patients confined to beds. The computing device or system receiving at least one of the detected flow property or the volume of the urine detected by the flow meter may determine trends in urine discharge by a patient. The computing device or system receiving at least one of the detected flow property or the volume of the urine detected by the flow meter may automatically push the received flow property and/or volume of the urine to other health applications, such as a hospital electronic medical records system. As shall be described in greater detail herein, the flow meter may be positioned in-line with a tube connecting a urine collection device and a urine collection container or may be secured or securable to a urine collection device between the urine collection device and a urine collection container. Systems and methods described herein may allow caregivers to bypass the steps of measuring urine discharged by a user and charting data pertaining to the volume of urine discharged by the user.

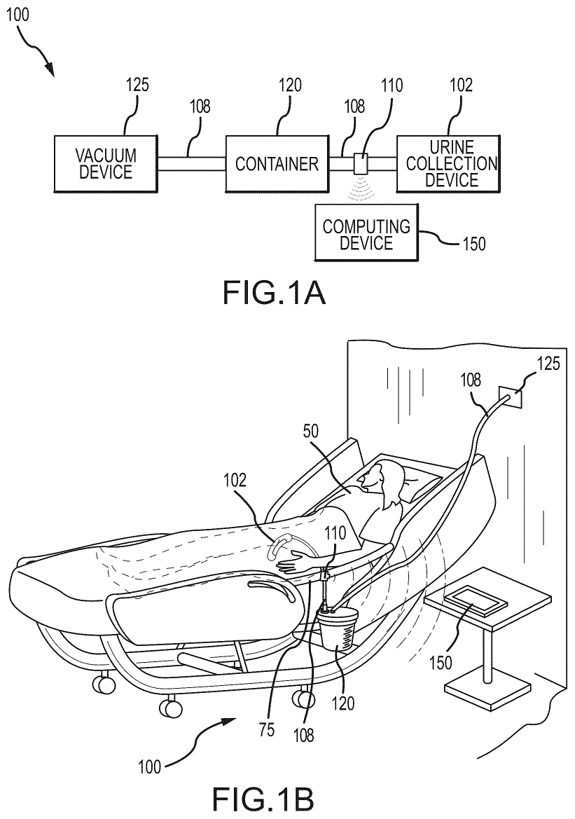

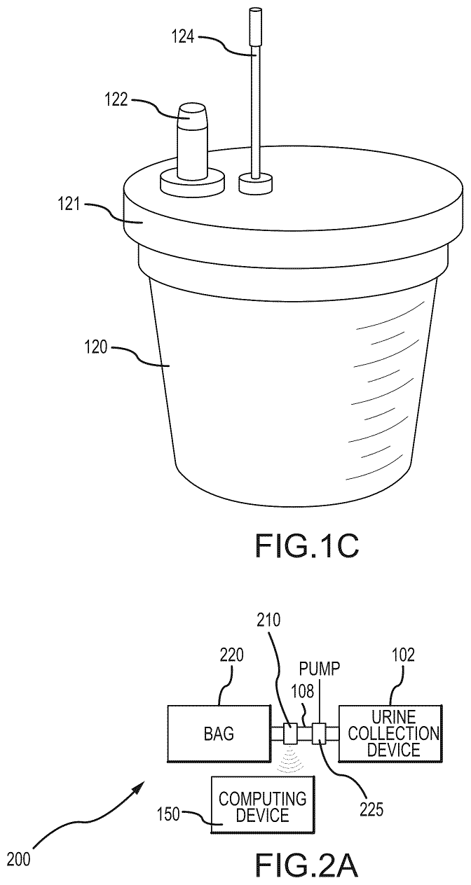

FIG. 1A is a block diagram of a urine collection system 100, according to an embodiment. The urine collection system 100 includes a fluid (e.g., urine) collection device 102, a conduit 108, a fluid (e.g., urine) storage container 120, a vacuum device 125, and a flow meter 110. The urine collection device 102 may include a male or female urine collection device. PCT International Application No. PCT/US2019/029616, for example, describes various embodiments of both male and female urine collection devices, the disclosure of which is incorporated in its entirety by this reference. Moreover, the urine collection device 102 may be interchangeable in the urine collection system 100 between different types, varieties, and sizes of male or female urine collection devices. The urine collection device 102 is configured to be positioned proximate or adjacent to a urethra of a user. Generally, the urine collection device 102 may include a surface sized to be positioned proximate or adjacent to the urethra and configured to wick urine or other fluids away from the user. Urine or other fluids may be wicked from the surface to a reservoir in the urine collection device 102.

The conduit 108 may include a flexible material such as plastic tubing (e.g., medical tubing). Such plastic tubing may include a thermoplastic elastomer, polyvinyl chloride, ethylene vinyl acetate, polytetrafluoroethylene, etc., tubing. In some embodiments, the conduit 108 may include silicone or latex. The urine collection device 102, the container 120, and the vacuum source 125 may be fluidly coupled to each other via one or more conduits 108. For example, urine collection device 102 may be operably coupled to one or more of the urine storage container 120 or the vacuum device 125 via the conduit 108. Fluid (e.g., urine or other bodily fluids) collected in the urine collection device 102 may be removed from the urine collection device 102 via the conduit 108 secured to the urine collection device 102. Suction force may be introduced into the chamber of the urine collection device 102 via the inlet of the conduit 108 responsive to suction (e.g., vacuum) force applied at the outlet of the conduit 108.

The suction force may be applied to the outlet of the conduit 108 by the vacuum device 125 either directly or indirectly. The suction force may be applied indirectly via the urine storage container 120. For example, the outlet of the conduit 108 may be connect to or disposed within the urine storage container 120 and an additional conduit 108 may extend from the urine storage container 120 to the vacuum device 125. Accordingly, the vacuum device 125 may apply suction to the urine collection device 102 via the urine storage container 120. The suction force may be applied directly via the vacuum device 125. For example, the outlet of the conduit 108 may be disposed within the vacuum device 125. An additional conduit 108 may extend from the vacuum device 125 to a point outside of the urine collection device 102, such as to the urine storage container 120. In such examples, the vacuum device 125 or other pump may be disposed between the urine collection device 108 and the urine storage container 120.

The urine storage container 120 is sized and shaped to retain a fluid therein. The urine storage container 120 may include a bag (e.g., drainage bag), a bottle or cup (e.g., collection jar), or any other enclosed container for storing bodily fluid(s) such as urine. In some examples, the conduit 108 may extend from the urine collection device 102 and attach to the urine storage container 120 at a first point therein. An additional conduit 108 may attach to the urine storage container 120 at a second point thereon and may extend and attach to the vacuum device 125. Accordingly, a vacuum (e.g., suction) may be drawn through urine collection device 102 via the urine storage container 120. Fluid, such as urine, may be drained from the urine collection device 102 using the vacuum device 125.

The vacuum device 125 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum device 125 may provide a vacuum or suction to remove fluid from the urine collection device 125. In some examples, the vacuum device 125 may be powered by one or more of a power cord (e.g., connected to a power socket), one or more batteries, or even manual power (e.g., a hand operated vacuum pump). In some examples, the vacuum device 125 may be sized and shaped to fit outside of, on, or within the urine collection device 102. For example, the vacuum device 125 may include one or more miniaturized pumps or one or more micro pumps.

The urine collection system 100 also includes a flow meter 110 configured to detect a flow property of the urine at least related to a volume of the urine pulled from the urine collection device 102 and passing through conduit 108 before entering the urine storage container 120. In some embodiments, the flow meter 110 is secured or securable between urine collection device 102 and the urine storage container 120 such that a first portion of the conduit 108 extends between the urine collection device 102 and the flow meter 110 and a second portion of the conduit 108 extends between the flow meter 110 and the urine storage container 120. In some embodiments, the flow meter 110 is secured or securable directly to the urine collection device 102, i.e. the conduit 108 extends between the flow meter 110 and the urine storage container, but a conduit or tubing is absent between the urine collection device 102 and the flow meter 110. In some embodiments, the flow meter 110 is secured or securable directly to the urine storage container 120, i.e. the conduit 108 extends between the urine collection device 102 and the flow meter 110, but a conduit or tubing is absent between the flow meter 110 and the urine storage container 120.

The flow meter 110 may include any suitable flow meter configured to detect a flow property of the urine that may be used to determine a volume of the urine passing through or within a predetermined proximity to the flow meter 110. The flow property detected by the flow meter 110 may include at least one of a volume, a mass, or a velocity of the urine passing through or proximate to the flow meter 110. In some embodiments, the flow meter may include a positive displacement flow meter configured to directly measure the actual volume of the urine passing through the flow meter. In some embodiments, the flow meter is configured to detect a mass or a velocity of the urine passing through or proximate to the flow meter 110, which mass or velocity may be translated (by at least one of the flow meter 110 or the computing device 150) into a volume or flow rate. The flow meter 110 may detect a duration of time during which urine is passing through or within a predetermined proximity to the flow meter 110. In some embodiments, the flow meter 110 or other computing device may then calculate that volume of the urine that has passed the flow meter 110 based on a known cross-sectional area or volume of the conduit 108 or the flow meter 110.

In some embodiments, the flow meter 110 may mechanically or ultrasonically detect the flow property of the urine passing between the urine collection device 102 and the urine collection container 120. For example, at least a portion of the flow meter 110 may be in line with the conduit 108 effective to allow the flow meter 110 to mechanically detect the flow property of the urine through the conduit 108. In some embodiments, at least a portion of the flow meter 110 extends into the conduit 108 effective to allow the flow meter 110 to mechanically detect the flow property of the urine through the conduit 108. The flow meter 110 may include at least one of a bi-rotor, a disc, a piston, a propeller, a paddle wheel, and/or a rotameter. In some embodiments the flow meter 110 may include laminar or venturi flow meter. In some embodiments, the flow meter 110 may be detachably or fixedly secured or securable to the outside of the conduit 108 and ultrasonically (i.e., via the Doppler effect) or electromagnetically detect the flow property of the urine through the conduit 108. Detachable securement of the flow meter 110 to the conduit 108 allows the flow meter to be reused with multiple conduits 108 and/or users. For example, a Doppler ultrasonic flow meter may be secured or securable outside of the conduit 108 and be reused on multiple urine collection systems. In some embodiments, the flow meter 110 may include system of valves configured to close off the fluid collection container 120 at a certain, predetermined volume and only allow that predetermined volume to be withdrawn from the urine collection device 102.

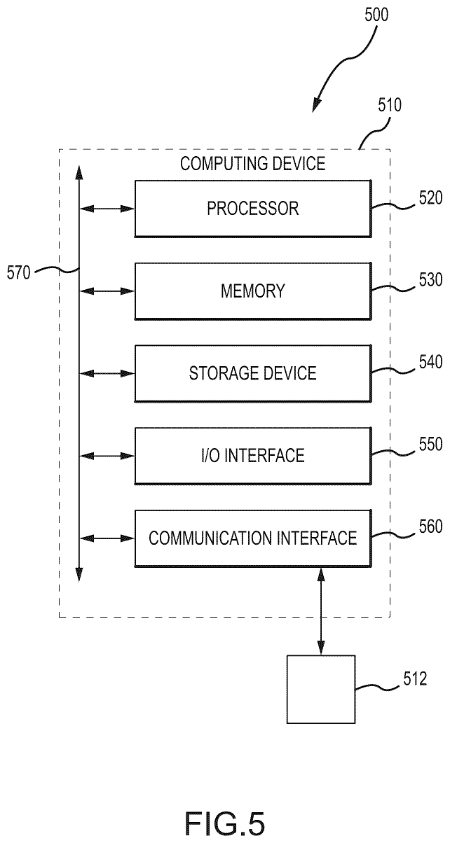

The flow meter 110 also may include a communication interface configured to wirelessly communicate to a computing device 150 separate or external to the flow meter 110. For example, the flow meter 110 may be configured to wirelessly communicate via BLUETOOTH or WI-FI to the computing device 150 data including at least one of the detected flow property or the volume of the urine discharged from the user and entering the fluid collection container 120. In some embodiments, the flow meter 110 is configured to wirelessly communicate the volumetric flow data to the computing device 150 in predetermined intervals or whenever urine flow is detected. The computing device 150 may include various computing devices configured to wirelessly communicate with the flow meter 110. For example, the computing device 150 may include a smartphone, a tablet computer, a desktop computer, or medical computing devices found in hospital or healthcare rooms. In some embodiments, the flow meter 110 is configured to wirelessly communicate the flow property of the urine to the computing device 150 which includes a software application configured to record the data including the flow property of the urine, display the flow property and/or volume of the urine discharged by the user 50, and/or determine and display trends and totals related to the flow property and/or volume of the urine discharged by the user. The computing device 150 may be located on the user, in a hospital room, in a home of the user, or combinations thereof. Additional details regarding the schematics and wireless communications of the flow meter 110 and the computing device 150 are provided below in reference to FIG. 5.

At least one of the flow meter 110 or the computing device 150 may be configured to automatically translate data of the flow property into a volume measurement, such as the volume of urine discharged by a user over a predetermined period of time (such as an hour or a day). Data including volume measurements transmitting to the computing device 150 may be recorded and/or pushed to an electronic medical record (EMR) system for future reference and consideration. At least one of the flow meter 110 or the computing device 150 may provide the volume of urine entering the urine collection device 102 in real-time using the data detected and/or communicated by the flow meter 110. The computing device 150 may transmit or signal an alert when a predetermined volume of fluid is discharged by the user. The computing device 150 may be configured to determine and provide trends in the volume of urine discharged by the user.

FIG. 1B shows a urine collection system 100 in use in a hospital room environment, according to an embodiment. Although the user 50 is shown lying down in FIG. 1B, in some embodiments, the user 50 may be ambulatory during use of the urine collection system 100. The urine collection device 102 shown in FIG. 1B includes a female urine collection device positioned adjacent or proximate to the urethra of the user 50. In some embodiments, the urine collection device 102 may include a male urine collection device positioned over a male urethra or having at least a portion of a penis inserted therein. The vacuum device 125 shown in FIG. 1B includes a wall-mounted device or wall-connected vacuum source. In some embodiments, the vacuum device 125 may include a portable or modular vacuum device or pump. The computing device 150 shown in FIG. 1B includes a bedside computing device or table computing device. In some embodiments, the computing device 150 may include any computing device configured to wirelessly communicate with the flow meter 110.

The flow meter 110 shown in FIG. 1B includes a flow meter attached to the conduit 108 and in-line with the conduit 108 such that a first portion of the conduit 108 extends between the urine collection device 102 and the flow meter 110 and a second portion of the conduit 110 extends between the flow meter 110 and the urine storage container 120. In some embodiments, the flow meter 110 may include any of the flow meters described above in any of the positions described above. The flow meter 110 also may be configured to detachably secure to a portion of a bed, such as an arm or frame 75 of the bed. The urine storage container 120 also may be configured to detachably secure to a portion of the bed, such as an arm or frame 75 of the bed.

In use, the user 50 may void or discharge urine into the urine collection device 102. The vacuum device 125 may pull a vacuum on the urine collection device 102 effective to pull urine discharged into the urine collection device 102 through the conduit 108 and into the urine storage container 120. As the urine is pulled from the urine collection device 102 and into the urine storage container 120, the flow meter 110 detects the flow property of the urine at least related to the volume of the urine passing from the urine collection device 102 to the urine storage container 120. The flow meter 110 may then wirelessly transmit or communicate to the computing device 150 data including the flow property of the urine at least related to the volume of the urine passing from the urine collection device 102 to the urine storage container 120.

FIG. 1C shows a urine storage container 120 used in a urine collection system 100, according to an embodiment. The urine storage container 120 may be substantially rigid effective to allow a vacuum to be pulled on the urine collection device 102 and the conduit 108 through the urine storage container 120. The urine storage container 120 may include a lid 121 having multiple ports. For example, the lid 121 may include a vacuum port 124 configured to secure to a conduit 108 in fluid communication with the vacuum device 125 such that the vacuum device 125 can pull a vacuum on the interior region of the urine storage container 120 through the vacuum port 124. The lid 124 may include a urine collection port 122 configured to secure to a conduit 108 in fluid communication with an interior region of the urine collection device 125 such that a vacuum pulled on the interior region of the urine storage container 120 pulls a vacuum on the interior region of the urine collection device 102 effective to pull urine in the urine collection device 102 through the conduit 108, pass the flow meter 110, and into the interior region of the storage container 120.

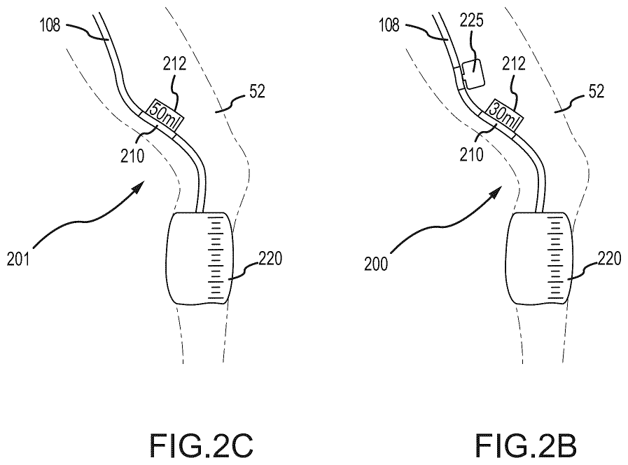

FIG. 2A is a block diagram of a urine collection system 200, according to an embodiment. Unless otherwise noted, the urine collection system 200 may include any aspect of the urine collection device 100 described above, such as the urine collection device 102, the conduit 108, and the computing device 150. The urine collection system 200 also may include a fluid storage bag 220, a flow meter 210, and a pump 225 positioned in-line with the conduit 108. The flow meter 210 is configured to wirelessly communicate with the computing device 150. The conduit 108 may fluidly connect the urine collection device 102 and the bag 220. The pump 225 may be positioned between the urine collection device 102 and the fluid storage bag 220. In some embodiments, the pump 225 is positioned between the urine collection device 102 and the flow meter 210. In some embodiments, the pump 225 is positioned between the flow meter 210 and the fluid storage bag 220.

The pump 225 of the urine collection system 200 may include any of a variety of pumps, such as a diaphragm pump. The pump 225 may include multiple flow rates that allow a user or wearer to select a desired or preferred flow rate. The pump 225 may include a removable or modular pump that may allow a user to remove the pump 225 and attach a different pump to the urine collection system 200. The pump 225 also may include a portable or modular pump that is transportable by a user or wearer. For example, the pump 225 may be sized to carry in a backpack or attach to the user or clothing of a user. The pump 225 may include a height of less than about 2.5 cm, a width of less than about 2.5 cm, and a depth of less than about 1.3 cm. The pump 225 also is configured to pull a vacuum in the conduit 108 to pull fluid from the urine collection device 102. The pump 225 also may be configured force fluid through the conduit 108 into the collection bag 220.

The urine collection system 200 also may include a power supply (not shown). The power supply may be a modular power supply that is detachable from the urine collection system 100 and replaceable with a similar or different power supply. For example, a user may be plan to engage in a longer ambulatory activity, and therefore desire a longer lasting power supply. A user also may desire to switch an initial power supply to a new power supply when the initial power supply is depleted. In some embodiments, the power supply is built into the pump 225 and/or the flow meter 210. That is, the pump 225 and/or the flow meter 210 may include a housing or compartment for the power supply. In some embodiments, the power supply may be external to the pump 225 and/or the flow meter 210, but may be electrically coupled to the pump 225 and/or the flow meter 210 such that the power supply may selectively power the pump 225 and/or the flow meter 210. The power supply may include one or more batteries, such as rechargeable batteries. The power supply also may include a wire and plug configured to plug into a wall outlet. In some embodiments, the power supply may include one or more rechargeable batteries and a wire and plug configured to charge the one or more rechargeable batteries.

The urine collection system 200 also includes a fluid storage bag 220. The fluid storage bag 220 may be a collapsible or expandable bag. For example, the fluid storage bag 220 may be generally flat when empty, but may expand when urine or other fluid is forced into the fluid storage bag 220. In some embodiments, the urine storage container 120 described above may replace the fluid storage bag 220 in the urine collection system 200.

FIG. 2B shows a portion of the urine collection system 200 in use, according to an embodiment. In some embodiments, the fluid storage bag 220 may be configured to secure to a portion of the body of the user, such as the leg 52. The fluid storage bag 220 may include straps or other elements configured to detachably secure the fluid storage bag to the user 50. The conduit 108 may be sized and dimensioned to extend between the urine collection device 102 (not shown in FIG. 2B) and the urine storage bag 220. For example, the conduit 108 may be sized and dimensioned to extend from the pubic region of the user 50 and down at least a portion of the leg 52 of the user 50 to the fluid storage bag 220 secured or securable to the leg 52.

The urine collection system 200 includes a flow meter 210. Unless otherwise noted, the flow meter 210 may include any aspect of the flow meter 110. The flow meter 210 may include a display 212 that displays a volume of urine detected by the flow meter 210 over a selected period of time. For example, the display 212 may display the volume of urine detected by the flow meter 210 until the flow meter 210 is reset to zero. In some embodiments, the display 212 is configured to display the volume of urine detected by the flow meter 210 during a period of time such as an hour, multiple hours, a day, multiple days, and so on. In some embodiments, the flow meter 210 may allow the user 50 or care worker to select the period of time elapsed before the volume displayed on the display 212 resets to zero. In some embodiments, the flow meter 110 may replace the flow meter 210.

In some embodiments, the pump 225 may be positioned in-line with the conduit 108 between urine collection device 102 and the flow meter 210, shown in FIG. 2B. In other embodiments, the pump 225 may be positioned in-line with the conduit 108 between the flow meter 210 and the fluid storage bag 220.

In use, the user 50 may void or discharge urine into the urine collection device 102. The pump 225 may pull urine from the urine collection device 102 through the conduit 108 and into the fluid storage bag 220. In some embodiments, the urine collection system 200 may be used with a female urine collection device. As the urine is pulled from the urine collection device 102 and into the fluid storage bag 220, the flow meter 210 detects the flow property of the urine at least related to the volume of the urine passing from the urine collection device 102 to the fluid storage bag 220. The flow meter 210 may then wirelessly transmit or communicate to the computing device 150 data including the flow property of the urine at least related to the volume of the urine passing from the urine collection device 102 to the fluid storage bag 220.

FIG. 2C shows an embodiment of a urine collection system 201, according to an embodiment. Unless otherwise noted, the urine collection system 201 may include any aspect of the urine collection system 200. In some embodiments, the urine collection system 201 may include a urine collection device 102 (not shown in FIG. 2C), the conduit 108, the flow meter 210, and the fluid storage bag 220, while the pump 225 may be absent from the urine collection system 201. In use, the user 50 may void or discharge urine into the urine collection device 102. Gravity may then pull urine from the urine collection device 102 through the conduit 108 and into the fluid storage bag 220. In some embodiments, the urine collection system 201 may be used with a male urine collection device 102. As the urine is pulled or otherwise forced from the urine collection device 102 and into the fluid storage bag 220, the flow meter 210 detects the flow property of the urine at least related to the volume of the urine passing from the urine collection device 102 to the fluid storage bag 220. The flow meter 210 may then wirelessly transmit or communicate to the computing device 150 data including the flow property of the urine at least related to the volume of the urine passing from the urine collection device 102 to the fluid storage bag 220.

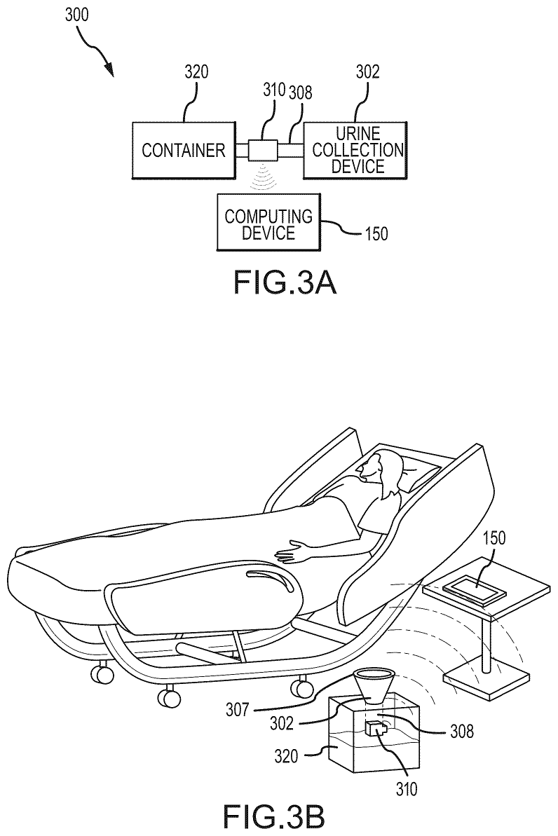

FIG. 3A is a block diagram of a urine collection system 300, according to an embodiment. Unless otherwise note, the urine collection system 300 can include any aspect of the urine collection systems 100, 200 described above, such as the computing device 150. The flow meter 310 is configured to wirelessly communicate with the computing device 150. The urine collection system 300 also may include a urine storage container 320 and a urine collection device 302 in fluid communication with the urine storage container 320. The flow meter 310 may be positioned between at least a portion of the urine collection device 302 and at least a portion of the fluid storage 320.

FIG. 3B shows the urine collection system 300 in use in a hospital room setting, according to an embodiment. In some embodiments, the urine collection system 300 may be used in a bathroom setting or a home room setting. The urine collection device 302 may include a funnel-shaped fluid or bowl-shaped urine collection device having wide end 307 and narrow end 308 distal to the wide end 307. In some embodiments, the urine collection device 302 may include a fluid collection seat. For example, a fluid collection seat may include a wide end 307 having a size that allows a user 50 to sit on the fluid collection seat to urinate into the fluid collection seat. The urine collection device 302 also may be sized and dimensioned to a man or woman to urinate into the urine collection device from a standing position.

The flow meter 310 is configured to wirelessly communicate with the computing device 150 and, unless otherwise noted, may include any aspect of the flow meters 110, 210. For example, although not shown in FIG. 3B, the flow meter 310 may include the display 212 of the flow meter 210. Moreover, the flow meter 310 may include be configured to detect flow properties of the urine at least related to the volume as described above in relation to the flow meters 110, 210. For example, the flow meter 310 may be configured to detect a flow property of the urine at least related to the volume of urine as urine passes or moves from the urine collection device 302 to the urine storage container 320.

The flow meter 310 may be fixedly or detachably secured or securable to a portion of the urine collection device 302. For example, the flow meter 310 may be fixedly or detachably secured or securable proximate to the narrow end 308 of the urine collection device 302. In some embodiments, the flow meter 310 is fixedly or detachably secured or securable to the narrow end 308 such that the urine collection device 302 does not extend beyond the flow meter 310. In some embodiments, the flow meter 310 is fixedly or detachably secured or securable to urine collection device 302 at least partially between the narrow end 308 and the wide end 307. In some embodiments, the flow meter 310 may be detachably secured or securable to the urine collection device 302 with one or more of a threaded fastening, clips, or friction fit.

The urine storage container 320 is configured to receive urine discharged by the user 50 into the urine collection device 302 after the flow meter 310 has detected the flow property of the urine at least related to the volume of the urine discharged into the urine collection device 302. At least a portion of the urine storage container 320 may be positioned below the urine collection device 302 such that gravity pulls the from the urine collection device 302 into the urine storage container 320. In some embodiments, the urine storage container includes one or more supports configured to support at least a portion of the urine collection device 302 when a user is sitting on and/or urinating in the urine collection device 302. The one or more supports may include a layer or one or more support bars through which at least a portion of the urine collection device 302 extends, such as the narrow end 308. The urine storage container 320 may be sized and dimensioned to allow a user or caregiver to carry or otherwise transport the urine storage container 320 to the toilet or disposal area after the user has discharged urine into the urine storage container 320 through the urine collection device 302.

In some embodiments, the urine storage container 320 includes a toilet (not shown). For example, the urine collection device 302 may be sized and dimensioned to position on conventional toilets within contact the water in the toilets. The urine collection device 302, then, may be sized and dimension to rest on a toilet seat and support a user sitting on the wide end 307 of the urine collection device 302 while the urine collection device 302 is resting on the toilet seat. Urine discharged into the urine collection device 302 resting on the toilet seat may flow to the narrow end 308 of the urine collection device and the flow property of the urine may be measured by the flow meter 310 as the urine is passing from the urine collection device 302 to the toilet.

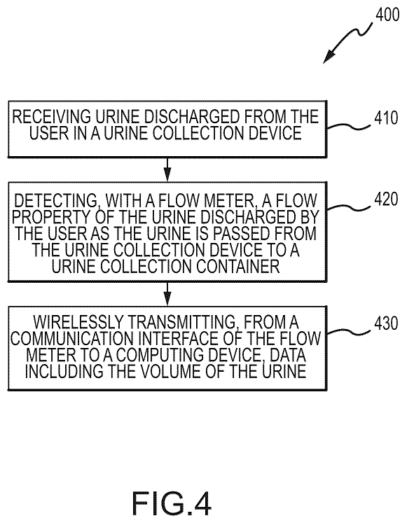

FIG. 4 is a flow diagram of a method 400 of determining a volume of urine discharged by a user, according to an embodiment. The method 400 may include an act 410 of receiving urine discharged from the user in a urine collection device. The method 400 also may include an act 420 of detecting, with a flow meter, a flow property of the urine at least related to a volume of the urine discharged by the user as the urine is passed from the urine collection device to a urine collection container. The method 400 also may include an act 430 of wirelessly transmitting, from a communication interface of the flow meter to a computing device, data including at least the flow property of the urine related to a volume of the urine.

The act 410 may include receiving urine discharged from the user in a urine collection device, such as urine collection device 102. Accordingly, in some embodiment, the act 410 may include receiving the urine discharged from the user in a urine collection device positioned proximate to a urethra of the user. In some embodiments, the method 400 also may include an act of pulling a vacuum on a first conduit in fluid communication with the urine collection device effective to draw urine from the urine collection device through the first conduit and into the urine collection container, with the flow meter being secured to the first conduit. In some embodiments, the method 400 may include an act of pulling a vacuum on the first conduit in fluid communication with the urine collection device and a second conduit in fluid communication with the first conduit and the urine collection container effective to draw the urine from the urine collection device through the first conduit, the flow meter, and the second conduit into the urine collection container, with the flow meter being secured to the second conduit between the first conduit and the second conduit.

In some embodiments, the act 410 may include receiving urine discharged from the user in the urine collection device 302. Accordingly, the act 410 may include receiving the urine discharged from the user in a funnel-shaped urine collection device having a wide end and a narrow end. In some embodiments, the method 400 also may include positioning the funnel-shaped urine collection seat on the urine collection container.

The act 420 may include detecting, with a flow meter, the flow property of the urine at least related to a volume of the urine discharged by the user as the urine is passed from the urine collection device to a urine collection container. In some embodiments, the act 420 includes detecting, with the flow meter secured to the narrow end of the funnel-shaped urine collection device, the flow property of the urine discharged by the user as the urine is passed from the urine collection device to the urine collection container. In some embodiments, the method 400 may include an act of regulating the volume of the urine passing through the flow meter. In some embodiments, the method 400 may include an act of displaying, on a display on the flow meter, the volume of the urine entering the urine collection container during a selected period of time.

The act 430 may include wirelessly transmitting, from a communication interface of the flow meter to a computing device, data including the flow property of the urine at least related to the volume of the urine. In some embodiments, the method 400 also includes an act of providing, with the computing device, the volume of urine entering the urine collection device in real-time using the data communicated by the flow meter. In some embodiments, the method 400 also includes an act of transmitting, with the computing device, the data communicated by the flow meter including the volume of the urine to an electronic medical record system.

The acts of the method 400 described above, including the acts 410, 420, and 430, are for illustrative purposes. For example, acts of the method 400, including the acts 410, 420, and 430, can be performed in different orders, split into multiple acts, modified, supplemented, or combined. In an embodiment, acts of the method 400, including the acts 410, 420, and 430, can be omitted from the method 400. Any of the acts of the method 400, including the acts 410, 420, and 430, can include using any of the urine collection systems disclosed herein.

Any of the example urine collection systems 100, 200, 201, 300 disclosed herein may be used to carry out any of the example methods 400 disclosed herein. FIG. 5 is a schematic of a controller 500 that may be included in any of the flow meters 110, 210, 310 or the computing device 150 described herein, according to an embodiment. The controller 500 may be configured to implement one or more acts any of the example methods disclosed herein, such as the act 430 of the method 400 of wirelessly transmitting, from a communication interface of the flow meter to a computing device, data including the flow property of the urine at least related to a volume of the urine.

The controller 500 includes at least one computing device 510. The at least one computing device 510 is an exemplary computing device that may be configured to perform one or more of the acts described above, such as the act 430 of the method 400. The computing device 510 can comprise at least one processor 520, memory 530, a storage device 540, an input/output (“I/O”) device/interface 550, and a communication interface 560. While an example computing device 510 is shown in FIG. 5, the components illustrated in FIG. 5 are not intended to be limiting of the controller 500 or computing device 510. Additional or alternative components may be used in some examples. Further, in some examples, the controller 500 or the computing device 510 can include fewer components than those shown in FIG. 5. For example, in some embodiments the controller 500 in the flow meter 110, 210, 310 may include only the communication interface 560 described below. Components of computing device 510 shown in FIG. 5 are described in additional detail below.

In some examples, the processor(s) 520 includes hardware for executing instructions (e.g., instructions for carrying out one or more portions of any of the methods disclosed herein), such as those making up a computer program. For example, to execute instructions, the processor(s) 520 may retrieve (or fetch) the instructions from an internal register, an internal cache, the memory 530, or a storage device 540 and decode and execute them. In particular examples, processor(s) 520 may include one or more internal caches for data such as flow property and flow rate tables. As an example, the processor(s) 520 may include one or more instruction caches, one or more data caches, and one or more translation lookaside buffers (TLBs). Instructions in the instruction caches may be copies of instructions in memory 530 or storage device 540. In some examples, the processor 520 may be configured (e.g., include programming stored thereon or executed thereby) to carry out one or more portions of any of the example methods or systems disclosed herein.

In some examples, the processor 520 is configured to perform any of the acts disclosed herein such as the act 430 in the method 400 or cause one or more portions of the computing device 510 or controller 500 to perform at least one of the acts disclosed herein. Such configuration can include one or more operational programs (e.g., computer program products) that are executable by the at least one processor 520. For example, the processor 520 may be configured to automatically transmit flow property at least related to a volume of the urine to the computing device 512. The computing device 512 may include the computing device 150 in urine collection systems 100, 200, 201, 300. The computing device 512 may include any aspect of the controller 500 described herein, such as one or more of a processor, a memory, a storage device, an I/O interface, and/or a communication interface. The computing device 512 can include one or more servers, one or more computers (e.g., desk-top computer, lap-top computer), or one or more mobile computing devices (e.g., smartphone, tablet, etc.).

The at least one computing device 510 (e.g., a server) may include at least one memory storage medium (e.g., memory 530 and/or storage device 540). The computing device 510 may include memory 530, which is operably coupled to the processor(s) 520. The memory 530 may be used for storing data, metadata, and programs for execution by the processor(s) 520. The memory 530 may include one or more of volatile and non-volatile memories, such as Random Access Memory (RAM), Read Only Memory (ROM), a solid state disk (SSD), Flash, Phase Change Memory (PCM), or other types of data storage. The memory 530 may be internal or distributed memory.

The computing device 510 may include the storage device 540 having storage for storing data or instructions. The storage device 540 may be operably coupled to the at least one processor 520. In some examples, the storage device 540 can comprise a non-transitory memory storage medium, such as any of those described above. The storage device 540 (e.g., non-transitory storage medium) may include a hard disk drive (HDD), a floppy disk drive, flash memory, an optical disc, a magneto-optical disc, magnetic tape, or a Universal Serial Bus (USB) drive or a combination of two or more of these. Storage device 540 may include removable or non-removable (or fixed) media. Storage device 540 may be internal or external to the computing device 510. In some examples, storage device 540 may include non-volatile, solid-state memory. In some examples, storage device 540 may include read-only memory (ROM). Where appropriate, this ROM may be mask programmed ROM, programmable ROM (PROM), erasable PROM (EPROM), electrically erasable PROM (EEPROM), electrically alterable ROM (EAROM), or flash memory or a combination of two or more of these. In some examples, one or more portions of the memory 530 and/or storage device 540 (e.g., memory storage medium(s)) may store one or more databases thereon. At least some of the databases may be used to store flow properties of the urine and/or flow volumes of the urine, as disclosed herein.

In some examples, flow properties of the urine may be stored in a memory storage medium such as one or more of the at least one processor 520 (e.g., internal cache of the processor), memory 530, or the storage device 540. In some examples, the at least one processor 520 may be configured to access (e.g., via bus 570) the memory storage medium(s) such as one or more of the memory 530 or the storage device 540. For example, the at least one processor 520 may receive and store the data (e.g., look-up tables) as a plurality of data points in the memory storage medium(s). The at least one processor 520 may execute programming stored therein adapted access the data in the memory storage medium(s) to automatically transmit a flow property at least related to a volume of the urine to the computing device 512. For example, the at least one processor 520 may access one or more flow property data tables in the memory storage medium(s) such as memory 530 or storage device 540.

The computing device 510 also includes one or more I/O devices/interfaces 550, which are provided to allow a user to provide input to, receive output from, and otherwise transfer data to and from the computing device 510. These I/O devices/interfaces 550 may include a mouse, keypad or a keyboard, a touch screen, camera, optical scanner, network interface, web-based access, modem, a port, other known I/O devices or a combination of such I/O devices/interfaces 550. The touch screen may be activated with a stylus or a finger.

The I/O devices/interfaces 550 may include one or more devices for presenting output to a user, including, but not limited to, a graphics engine, a display (e.g., a display screen or monitor), one or more output drivers (e.g., display drivers), one or more audio speakers, and one or more audio drivers. In certain examples, I/O devices/interfaces 550 are configured to provide graphical data to a display for presentation to a user. The graphical data may be representative of one or more graphical user interfaces and/or any other graphical content as may serve a particular implementation.

The computing device 510 can further include a communication interface 560. The communication interface 560 can include hardware, software, or both. The communication interface 560 can provide one or more interfaces for communication (such as, for example, packet-based communication) between the computing device 510 and one or more additional computing devices 512 or one or more networks. For example, communication interface 560 may include a network interface controller (NIC) or network adapter for communicating with an Ethernet or other wire-based network or a wireless NIC (WNIC) or wireless adapter for communicating with a wireless network, such as a WI-FI.

Any suitable network and any suitable communication interface 560 may be used. For example, computing device 510 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, one or more portions of controller 500 or computing device 510 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination thereof. Computing device 510 may include any suitable communication interface 560 for any of these networks, where appropriate.

The computing device 510 may include a bus 570. The bus 570 can include hardware, software, or both that couples components of computing device 510 to each other. For example, bus 570 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination thereof.

It should be appreciated that examples of acts described herein, such as the act 430 in the method 400 may be performed by and/or at the computing device 510

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

What is claimed is:

1. A urine collection system, comprising:
a urine collection device configured to collect urine discharged from a user;
a urine collection container in fluid communication with the urine collection device and sized and dimensioned to receive the urine discharged from the user; and
a flow meter having a communication interface and positioned between the urine collection container and the urine collection device, wherein the flow meter is configured to detect a flow property of the urine at least related to a volume of the urine entering the urine collection container, and the communication interface is configured to wirelessly communicate to a computing device at least one of the detected flow property or the volume of the urine;
wherein the flow meter is configured to detect the volume of the urine entering the urine collection container during a selected period of time and the communication interface is configured to wirelessly communicate to the computing device the volume of the urine entering the urine collection container.

2. The urine collection system of claim 1, wherein the flow meter is configured to perform at least one of:
detecting the flow property of the urine at least related to the volume of the urine entering the urine collection container with a mechanical detector;
detecting the flow property of the urine at least related to the volume of the urine entering the urine collection container with an ultrasonic detector; or
detecting at least one of a volume, a mass, or a velocity of the urine passing the flow meter.

3. The urine collection system of claim 2, further comprising the computing device, the computing device being configured to perform at least one of:
providing the volume of the urine entering the urine collection device in real-time using the at least one of the detected flow property or the volume of the urine communicated by the flow meter; or
transmitting the at least one of the detected flow property or the volume of the urine communicated by the flow meter to an electronic medical record system.

4. The urine collection system of claim 1, further comprising a first conduit in fluid communication with the urine collection device, wherein the flow meter is secured or securable to the first conduit and configured to detect the flow property of the urine passing through the first conduit before entering the urine collection container.

5. The urine collection system of claim 1, wherein:
the urine collection device includes a funnel-shaped urine collection device having a wide end and a narrow end; and
the flow meter is secured or securable to the narrow end of the funnel-shaped urine collection device.

6. A method of determining a volume of urine discharged by a user, the method comprising:
receiving the urine discharged from the user in a urine collection device;
detecting, with a flow meter, a flow property of the urine at least related to a volume of the urine discharged by the user as the urine is passed from the urine collection device to a urine collection container;
determining, with the flow meter, the volume of the urine entering the urine collection container during a selected period of time; and
wirelessly transmitting, from a communication interface of the flow meter to a computing device, the volume of the urine entering the urine collection container during the selected period of time.

7. The method of claim 6, further comprising pulling a vacuum on a first conduit in fluid communication with the urine collection device effective to draw the urine from the urine collection device through the first conduit and into the urine collection container, wherein the flow meter is secured to a first conduit in fluid communication with the urine collection device and configured to detect the volume of the urine of the urine passing through the first conduit before entering the urine collection container.

8. The method of claim 6, further comprising regulating the volume of the urine passing through the flow meter.

9. The method of claim 6, wherein receiving urine discharged from the user in a urine collection device includes receiving the urine discharged from the user in the urine collection device positioned proximate to a urethra of the user.

10. The method of claim 6, wherein:
receiving urine discharged from the user in a urine collection device includes receiving the urine discharged from the user in a funnel-shaped urine collection device having a wide end and a narrow end; and
detecting, with a flow meter, a flow property of the urine at least related to a volume of the urine discharged by the user as the urine is passed from the urine collection device to a urine collection container includes detecting, with the flow meter secured to the narrow end of the funnel-shaped urine collection device, the flow property of the urine at least related to the volume of the urine discharged by the user as the urine is passed from the urine collection device to the urine collection container.

11. The method of claim 6, further comprising providing, with the computing device, the volume of the urine entering the urine collection device in real-time using the at least one of the detected flow property or the volume of the urine communicated by the flow meter.

12. The method of claim 6, further comprising transmitting, with the computing device, the at least one of the detected flow property or the volume of the urine communicated by the flow meter including the volume of the urine to an electronic medical record system.

13. A urine collection system, comprising:
a urine collection device configured to collect urine discharged from a user;
a urine collection container in fluid communication with the urine collection device and sized and dimensioned to receive the urine discharged from the user;
a first conduit in fluid communication with the urine collection device, configured to detect the flow property of the urine passing through the first conduit before entering the urine collection container;
a flow meter having a communication interface and secured or securable to the first conduit between the urine collection container and the urine collection device, wherein the flow meter is configured to detect a flow property of the urine passing through the first conduit before entering the urine collection container, and the communication interface is configured to wirelessly communicate to a computing device at least one of the detected flow property or the volume of the urine.

14. The urine collection system of claim 13, wherein the flow meter is configured to regulate the volume of the urine passing through the flow meter.

15. The urine collection system of claim 14 further comprising one or more valves associated with the first conduit configured to regulate the volume of urine passing through the first conduit, the one or more valves being configured to close and prevent the urine from passing through the first conduit when a predetermined volume of the urine has been collected in the urine collection container.

16. The urine collection system of claim 13, wherein the flow meter is position in-line with at least the first conduit.

17. The urine collection system of claim 13, wherein the flow meter is detachably secured or securable to the first conduit.

18. The urine collection system of claim 13, wherein the flow meter is configured to detect the volume of the urine entering the urine collection container during a selected period of time and the communication interface is configured to wirelessly communicate to the computing device the volume of the urine entering the urine collection container.

19. The urine collection system of claim 18, wherein the flow meter is configured to perform at least one of:
detecting the flow property of the urine at least related to the volume of the urine entering the urine collection container with a mechanical detector;
detecting the flow property of the urine at least related to the volume of the urine entering the urine collection container with an ultrasonic detector; or
detecting at least one of a volume, a mass, or a velocity of the urine passing the flow meter.

20. The urine collection system of claim 19, further comprising the computing device, the computing device being configured to perform at least one of:
providing the volume of the urine entering the urine collection device in real-time using the at least one of the detected flow property or the volume of the urine communicated by the flow meter; or
transmitting the at least one of the detected flow property or the volume of the urine communicated by the flow meter to an electronic medical record system.
